(19) United States
(12) Patent Application Publication
(10) Pub. No.: US 2011/0311555 A1
(43) Pub. Date: Dec. 22, 2011

(54) Tc11 as a Transcriptional Regulator

(75) Inventors: Carlo M. Croce, Columbus, OH (US); Yuri Pekarsky, Columbus, OH (US)

(73) Assignee: THE OHIO STATE UNIVERSITY, Columbus, OH (US)

(21) Appl. No.: 13/129,881

(22) PCT Filed: Nov. 19, 2009
(86) PCT No.: PCT/US09/65072

§ 371 Date: Jun. 9, 2011

Related U.S. Application Data
(60) Provisional application No. 61/116,786, filed on Nov. 21, 2008.

(51) Int. Cl. A61K 39/395; A61P 35/02; C07K 2/00; C07K 16/28; A61K 38/02

(52) U.S. Cl. 424/173.1; 530/389.6; 514/19.6; 530/350

(57) ABSTRACT

Methods and compositions for the diagnosis, prognosis and/or treatment of B cell chronic lymphocytic leukemia associated diseases are disclosed.

### CROSS-REFERENCE TO RELATED APPLICATIONS

This application claims the benefit of U.S. Provisional Application No. 61/116,786 filed Nov. 21, 2008, the entire disclosure of which is expressly incorporated herein by reference.

### STATEMENT REGARDING FEDERALLY SPONSORED RESEARCH

This invention was made with government support under P01 CA081534, awarded by National Cancer Institute Grant. The government has certain rights in this invention.

### TECHNICAL FIELD AND INDUSTRIAL APPLICABILITY OF THE INVENTION

This invention relates generally to the field of molecular biology. More particularly, it concerns methods for inhibiting development of mature B cell leukemia in a subject by inhibiting deregulation of Tcl1 in cells in the subject.

Certain aspects of the invention include application in diagnostics, therapeutics, and prognostics of B cell lymphocytic leukemia associated disorders.

### BACKGROUND OF THE INVENTION

There is no admission that the background art disclosed in this section legally constitutes prior art.

The lymphocytes of B cell chronic lymphocytic leukemia (B-CLL) are mostly resting cells with mature appearance and the B220+CD5+ phenotype. The T cell leukemia/lymphoma 1 (TCL1) oncogene was discovered as a target of chromosomal translocations and inversions at 14 g 31.2 in T cell prolymphocytic leukemias.

Transgenic mice overexpressing TCL1 in B cells develop the aggressive form of B-CLL and aggressive human B-CLLs overexpress Tcl1, indicating that deregulation of TCL1 is critically important in the pathogenesis of the aggressive form of B-CLL. Previously, the inventors herein have demonstrated that Tcl1 is a coactivator of the Akt oncoprotein, a critical antiapoptotic molecule in T cells. More recently, it has been reported that transgenic mice expressing constitutively active myristylated Akt in T cells develop T cell leukemias. These results suggest that Akt may be responsible for Tcl1-mediated lymphomagenesis in T cells. Akt could be robustly activated in mouse B cells by homozygous deletion of Pten. Surprisingly, these mice did not develop B cell malignancies, suggesting that Tcl1 deregulation in B cells causes B-CLL by mechanisms other than Akt activation.

Recent studies of transgenic mouse models demonstrated the importance of the NF-κB pathway in B-CLL. For example, transgenic expression of a proliferation-inducing TNF ligand (APRIL), a member of the TNF superfamily involved in NF-κB activation, resulted in significant expansions of B220+CD5+ cells.

In spite of considerable research into therapies to treat these diseases, they remain difficult to diagnose and treat effectively, and the mortality observed in patients indicates that improvements are needed in the diagnosis, treatment and prevention of these disease.

### SUMMARY OF THE INVENTION

In a first broad aspect, there is described herein a method for inhibiting development of mature B cell leukemia in a subject, comprising inhibiting deregulation of Tcl1 in cells in the subject.

In another broad aspect, there is provided herein a method for inhibiting development of mature B cell chronic leukemia (B-CLL) in a subject, comprising: inhibiting over-expression of Tcl1 in cells in the subject by one or more of: i) inhibiting the NF-κB pathway in the cells, and ii) activating activator protein 1 (AP-1) in the cells.

In another broad aspect, there is provided herein a method of treating a subject with a B cell chronic lymphocytic leukemia associated disease, comprising: administering a therapeutically effective amount of a composition capable of inhibiting overexpression of T cell leukemia/lymphoma 1 (Tcl1) by one or more of: i) inhibiting the NF-κB pathway in the cells, and ii) activating activator protein 1 (AP-1) in the cells.

In another broad aspect, there is provided herein a method of treating a B cell chronic lymphocytic leukemia (B-CLL) associated disease in a subject, comprising: determining the amount of at least Tcl1 expressed in cells in the subject, relative to control cells Tcl1; and altering the amount of Tcl1 expressed in the subject by administering to the subject an effective amount of at least one compound for inhibiting expression of Tcl1 by one or more of: i) inhibiting the NF-κB pathway in the cells, and ii) activating activator protein 1 (AP-1) in the cells, such that proliferation of the B-CLL associated disease in the subject is inhibited.

In another broad aspect, there is provided herein a method of assessing the effectiveness of a therapy to prevent, diagnose and/or treat a B cell chronic lymphocytic leukemia associated disease, comprising: subjecting an animal to a therapy whose effectiveness is being assessed, and determining the level of effectiveness of the treatment being tested in treating or preventing a B cell chronic lymphocytic leukemia associated disease, by evaluating at least one biomarker for Tcl 1.

In certain embodiments, the candidate therapeutic agent comprises one or more of: pharmaceutical compositions, nutraceutical compositions, and homeopathic compositions. Also, in certain embodiments, the therapy being assessed is for use in a human subject.

In another broad aspect, there is provided herein a use of an agent that interferes with a B cell chronic lymphocytic leukemia associated disease response signaling pathway, for the manufacture of a medicament for treating, preventing, reversing or limiting the severity of a B cell chronic lymphocytic leukemia associated disease complication in an individual, wherein the agent comprises at least one biomarker for Tcl 1 .

In another broad aspect, there is provided herein a method of treating, preventing, reversing or limiting the severity of a B cell chronic lymphocytic leukemia associated disease complication in an individual in need thereof, comprising: administering to the individual an agent that interferes with at least a B cell chronic lymphocytic leukemia associated disease response cascade, wherein the agent comprises at least one biomarker for Tcl1 which: i) inhibits the NF-κB pathway in the cells, and/or ii) activates activator protein 1 (AP-1) expression in the cells.

In another broad aspect, there is provided herein a use of an agent that interferes with at least a B cell chronic lymphocytic leukemia associated disease response cascade, for the manufacture of a medicament for treating, preventing, reversing or limiting the severity of a cancer-related disease complication in an individual, wherein the agent comprises at least one biomarker for Tcl1 which: i) inhibits the NF-κB pathway in the cells, and/or ii) activates activator protein 1 (AP-1) expression in the cells.

In another broad aspect, there is provided herein an antibody which binds to an epitope on Tcl1, wherein the antibody modulates at least one of: an interaction between the epitope and activator protein 1 (AP-1). In another broad aspect, there is provided herein a pharmaceutical composition comprising such antibody.

In another broad aspect, there is provided herein a method of treating a B-CLL disease state in which the activity of activator protein 1 (AP-1) is altered in a mammal, comprising administering to the mammal a therapeutically effective amount of an antibody capable of binding to an epitope on a Tcl 1 protein, thereby modulating a Tcl1 enhanced activity of the activator protein 1 (AP-1).

In another broad aspect, there is provided herein a method of treating a B-CLL disease state in which the activity of activator protein 1 (AP-1) is altered in a mammal, comprising: administering to the mammal a therapeutically effective amount of a peptide fragment of activator protein 1 (AP-1), wherein the peptide fragment binds to the activator protein 1 (AP-1), thereby modulating a Tcl1 enhanced kinase activity of the activator protein 1 (AP-1).

In another broad aspect, there is provided herein a compound comprising a Tcl 1 mimic, wherein the Tcl1 mimic binds to an activator protein 1 (AP-1) in any cell and is functionally active in mimicking a Tcl 1 enhanced activation of the activator protein 1 (AP-1).

In another broad aspect, there is provided herein a method of treating a disease state in which the activity of activator protein 1 (AP-1) is altered in a mammal, comprising administering to the mammal a therapeutically effective amount of a Tcl1 mimic, wherein the Tcl 1 mimic binds to the activator protein 1 (AP-1), thereby activating a Tcl1 enhanced kinase activity of the activator protein 1 (AP-1).

In another broad aspect, there is provided herein a compound comprising a Tcl 1 antagonist, wherein the Tcl1 antagonist binds to activator protein 1 (AP-1) in any cell and is functionally active in modulating a Tcl 1 enhanced activation of the activator protein 1 (AP-1).

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1C: Tcl 1 activates NF-κB-dependent transcription:

FIG. 1A: Chromatograms of sequences surrounding T381, E40D, R52H mutations obtained from sequencing of buccal swab constitutional DNA, B-CLL DNA, and results of RT-PCR (for T381 mutant) using RNA from B-CLL cells.

FIG. 1B: Tcl1 activates NF-κB. NIH 3T3 cells were cotransfected with 50 ng of pNF-kB-Luc reporter and 50 ng of pRL-TK Renilla reporter constructs. In addition, 1.5 μg of CMV5-empty vector, or a combination of 0.75 μg of CMV5-empty vector and 0.75 μg of CMV5-Tcl1 WT, or CMV5-Tcl 1 T381 constructs were used. Five nanograms of pFC-MEKK were added where indicated. Cells were treated with 200 nmol/L of Wortmannin overnight, where indicated. The normalized promoter activity of pNF-kB-Luc in NIH 3T3 cells transfected with CMV5-empty vector was set as 1.

FIG. 1C: Tcl 1 interacts with p300. (Upper) Some 293 cells were cotransfected with p300-HA and Omni-Fhit or p300-HA and Omni-Tcl1 constructs. After lysis, immunoprecipitations were carried out with anti-HA, IgG, or anti-omni antibodies. Western blot analysis was carried out as indicated. (Lower) Daudi cells were lysed and immunoprecipitations were carried out with anti-Tcl1 antibody, IgG, or anti-p300 antibody. Unlabeled higher band in the Tcl1 panel represents IgG. Western blot analysis was carried out as indicated.

FIGS. 2A-2G: Tcl 1 inhibits AP-1 activity:

FIG. 2A: Some 293 cells were cotransfected with 500 ng of pAP-1-Luc reporter and 50 ng of pRL-TK Renilla reporter constructs. In addition, 1.5 μg of CMV5-empty vector, CMV5-Tcl 1 WT, or mutant constructs and 2.5 ng of pFC-MEKK (where indicated) were used. Cells were treated with 200 nmol/L of Wortmannin overnight, where indicated. The normalized promoter activity of pAP-1-Luc in HEK293 cells transfected with CMV5-empty vector was set as 1.

FIG. 2B: Same as in FIG. 2A, except instead of pFC-MEKK construct, 5 ng of c-Fos-V5, c-Jun, JunB, or combinations of 5 ng of c-Fos-V5 and S ng of c-Jun or JunB were added, as indicated.

FIG. 2C: Some 293 cells were cotransfected with c-Fos-V5 and CMV5-Tcl1 WT or c-Fos-V5 and CMV5-Tcl 1 T381 constructs. After lysis, immunoprecipitations were carried out with anti-c-Fos, IgG, or anti-TcI1 antibodies. Western blot analysis was carried out as indicated.

FIGS. 2D-2F: Some 293 cells were cotransfected with myc-Tcl1 T381 or myc-Fhit with c-Fos-V5 (FIG. 2D), c-Jun-HA (FIG. 2E), or JunB (FIG. 2F), as indicated. After lysis, immunoprecipitations were carried out with anti-myc, IgG, and anti-c-Fos (FIG. 2D), anti-HA (FIG. 2E), and anti-JunB (FIG. 2F) antibodies as indicated. Western blot analysis was carried out with the indicated antibodies.

FIG. 2G: Some 293 cells were transfected with myc-Tcl1 and treated with 50 ng/mL PMA and 1 μg/mL ionomycin to increase endogenous c-Jun expression, 2 h before lysis. Immunoprecipitations were carried out with anti-c-Jun, IgG, or anti-myc antibodies.

FIG. 2H: Daudi cells were treated with 50 ng/mL PMA and 1 μg/mL ionomycin 2 h before lysis. Immunoprecipitations were carried out with anti-Tcl1, IgG, or anti-c-Jun antibodies.

FIG. 3: Intracellular localization of c-Jun, c-Fos, and Tcl 1. Some 293 cells were cotransfected with c-Jun-HA, c-Fos-V5, and Omni-Tcl1 constructs. Sixteen hours later, cells were fixed, permeabilized, and immunostained with rat anti-HA, mouse anti-c-Fos, and rabbit anti-omni antibodies. Secondary goat anti-rat Alexa Fluor 647, goat anti-mouse Alexa Fluor 546, and goat anti-rabbit Alexa Fluor 488 antibodies were used to visualize intercellular location of c-Jun (blue), c-Fos (red), and Tcl 1 (green). Colocalization of c-Fos and Tcl1 is shown in yellow.

FIGS. 4A-4: Tcl 1 inhibits MEKK1-mediated cell death:

FIG. 4A: Some 293 cells were transfected with 1.5 μg of pCMV5-empty vector, 0.5 μg of pFC-MEKK and 1 μg of pCMV5-empty, pCMV5-Tcl1 WT, or pCMV5-Tcl 1 T38I constructs. Western blot analysis was carried out as described herein.

FIGS. 4B-4C: Some 293 cells were transfected with 1.5 μg of pCMVS-empty vector, or 0.5 μg of pFC-MEKK and 1 μg of pCMV5-empty or pCMV5-Tcl1 WT constructs. Sixteen hours later, cells were fixed, permeabilized, and stained with Hoechst 33342.

FIG. 4B: Percentage of apoptotic cells. For each transfection at least 20 fields were selected for counting the percentage of dead cells (indicated by fragmented nucleus).

FIG. 4C: Results of the same experiment were visualized by using confocal microscopy.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention is based, at least in part, on the inventors' discovery that Tcl1 functions as a transcriptional regulator and is directly involved in the pathogenesis of chronic lymphocytic leukemia (CLL).

B cell chronic lymphocytic leukemia (B-CLL) is the most common human leukemia. Deregulation of the T cell leukemia/lymphoma 1 (TCL1) oncogene in mouse B cells causes a CD5-positive leukemia similar to aggressive human B-CLLs. To examine the mechanisms by which Tcl1 protein exerts oncogenic activity in B cells, the inventors herein investigated the effect of Tcl1 expression on NF-κB and activator protein 1 (AP-1) activity.

It is now shown herein that Tcl1 physically interacts with c-Jun, JunB, and c-Fos and inhibits AP-1 transcriptional activity. Additionally, Tcl1 activates NF-κB by physically interacting with p300/CREB binding protein.

The TCL1 gene was sequenced in 600 B-CLL samples and 2 heterozygous mutations were found: T38I and R52H. It is to be noted that both mutants showed gain of function as AP-1 inhibitors. The results indicate that Tcl1 overexpression causes B-CLL by directly enhancing NF-κB activity and inhibiting AP-1.

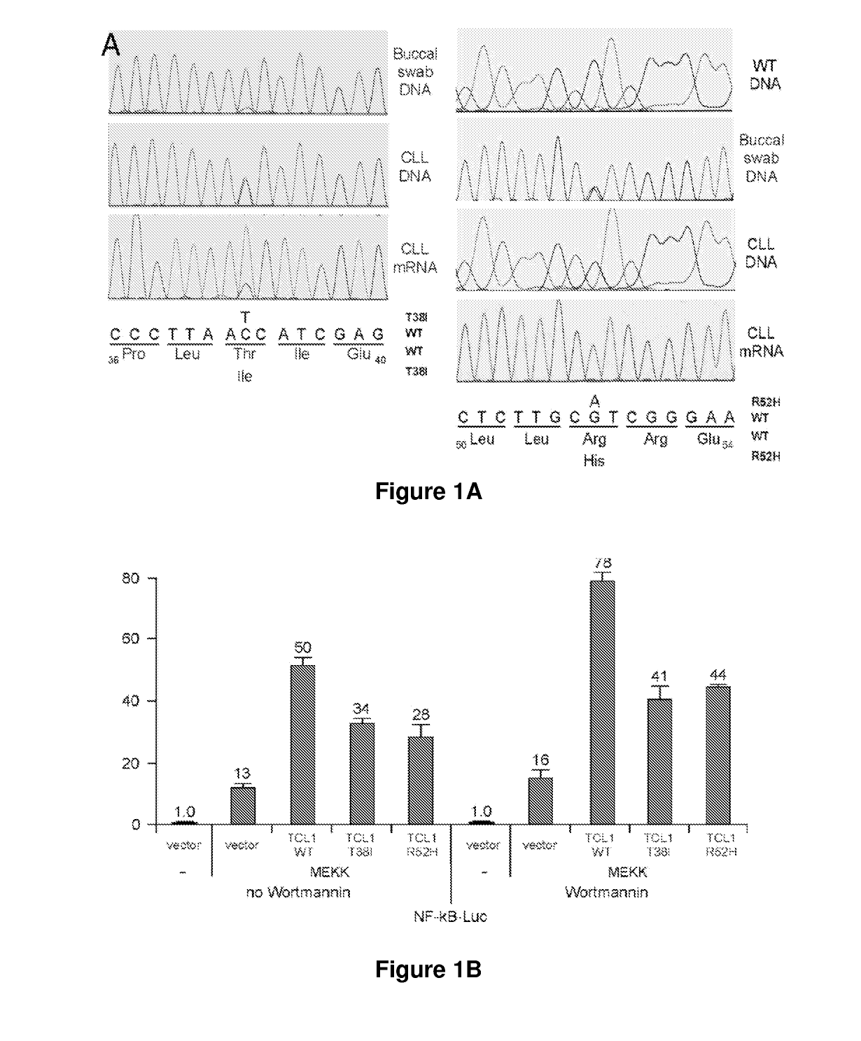

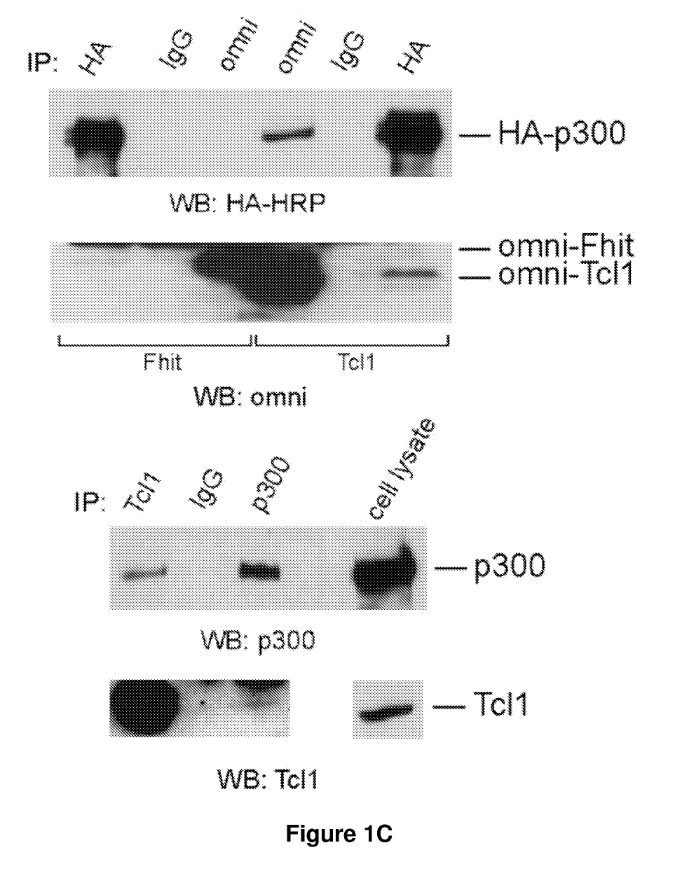

B-CLL-specific gain-of-function Tcl1 mutants were developed. The TCL1 gene in 600 B-CLL samples was sequenced. Sequencing analysis of all coding TCL1 exons resulted in the identification of 2 heterozygous mutations resulting in amino acid substitutions, T38I and R52H (FIG. 1A).

The normal buccal swab DNA of the first patient did not show the T38I mutation (FIG. 1A). The R52H mutation was also present in the matched normal buccal swab DNA (FIG. 1A Right), showing a constitutional variation. The RT-PCR results showed that the T38I mutant TCL1 mRNA was the major expressed allele in the B-CLL of origin, accounting for 80% of the TCL1 mRNA, and the R52H allele was the only allele expressed (FIG. 1A).

To determine whether Tcl 1 expression affects the transactivating activity of NF-κB, a system based on the ability of mitogen-activated protein kinase kinase 1 (MEKK1) was used to activate an NF-κB reporter construct, pNF-κB-Luc expressing luciferase under the control of an NF-κB-responsive element.

NIH 3T3 cells were transfected with the constructs indicated in FIG. 1B. FIG. 1B shows that Tcl11 activated NF-κB activity about 4-fold (50 versus 13), whereas the 2 mutants activated activity 2- to 3-fold.

Since Tcl1 is a coactivator of Akt, it was possible that this NF-κB activation is caused by Akt activation by Tcl 1. To eliminate this possibility the same experiment was performed in the presence of wortmannin, a PI3-kinase inhibitor (wortmannin completely inhibits Akt activity).

FIG. 1B shows that wortmannin did not affect the ability of Tcl 1 to activate NF-KB; in the presence of wortmannin Tcl 1 expression activated NF-κB>4-fold (78 versus 16), whereas the expression of Tcl1 mutants resulted in 2.5- to 3-fold activation.

In addition, wild type (WT) Tcl1 and T38I mutant did not show any difference in coimmunoprecipitation experiments with Akt (data not shown). These data show that Tcl 1 activates NF-κB by a mechanism independent of Akt.

To elucidate molecular mechanisms of this activation, coimmunoprecipitations between Tcl1 and NF-κB1, NF-κB2, RelA, RelB, and c-Rel by using cotransfections in 293 cells were carried out. No evidence of physical interactions between Tcl 1 and members of the NF-κB family was found (data not shown).

The transcriptional activator CREB binding protein/p300 is a ubiquitous nuclear transcription factor involved in transactivation mediated by several signaling pathways, including the NF-κB pathway. Because p300 is a coactivator of NF-κB, the inventors herein investigated whether Tcl 1 interacts with p300. First, coimmunoprecipitation experiments were carried out, cotransfecting tagged Tcl 1 and p300 constructs into 293 cells.

FIG. 1C-Upper shows that p300 was coimmunoprecipitated with Tcl 1, whereas Tcl1 was detected in p300 immune complexes. No coimmunoprecipitation was detected between p300 and Fhit, used as a negative control.

To prove that the interaction detected is not the result of overexpression of the 2 proteins, coimmunoprecipitation experiments were carried out in Daudi Burkitt lymphoma cells showing moderate levels of Tcl1 expression.

FIG. 1C-Lower shows that p300 was detected in Tcl1 immune complexes, whereas Tcl 1 was coimmunoprecipitated with p300. This shows that Tcl 1 induces NF-κB-dependent transcription by interacting with p300, perhaps changing its conformation and enhancing its ability to function as an NF-κB coactivator.

The results indicate that Tcl 1 mutants activate NF-κB-dependent transcription to a lesser extent than WT Tcl1 (-˜3-fold versus 4-fold). Activation of NF-κB is now believed by the inventors herein to be important in the pathogenesis of B-CLL. Also, the inventors herein now show that the Tcl1 mutants do not exhibit gain of function in the activation of NF-κB. In addition, the Tcl 1 T38I mutant protein was similar to WT Tcl 1 in coimmunoprecipitation experiments with p300 (data not shown).

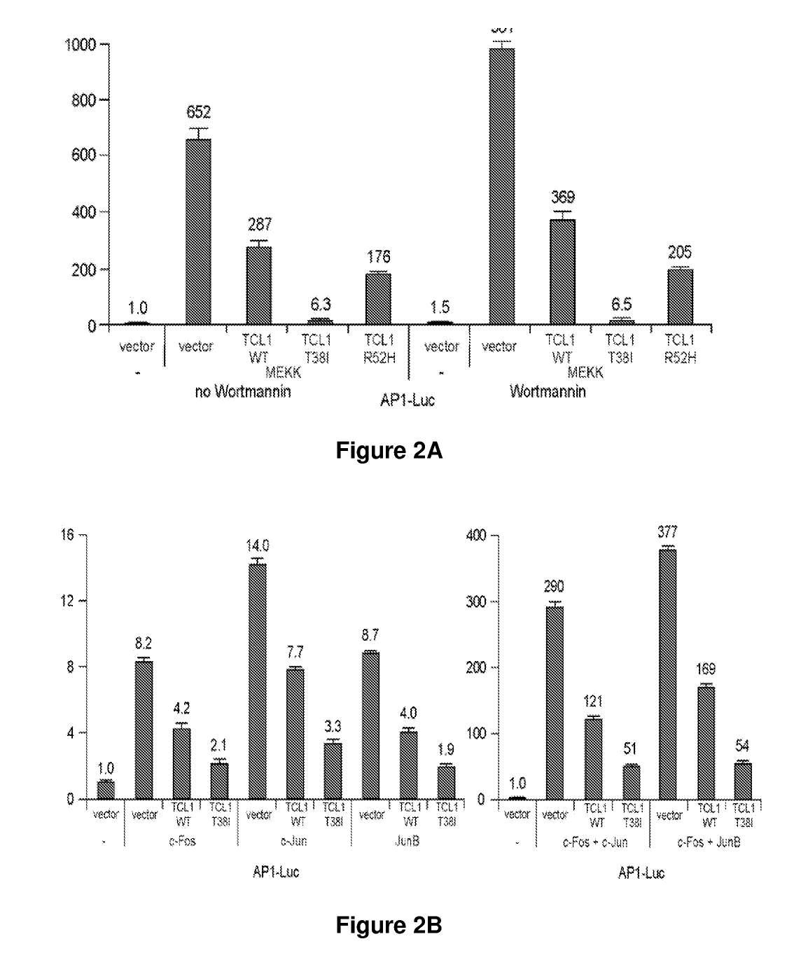

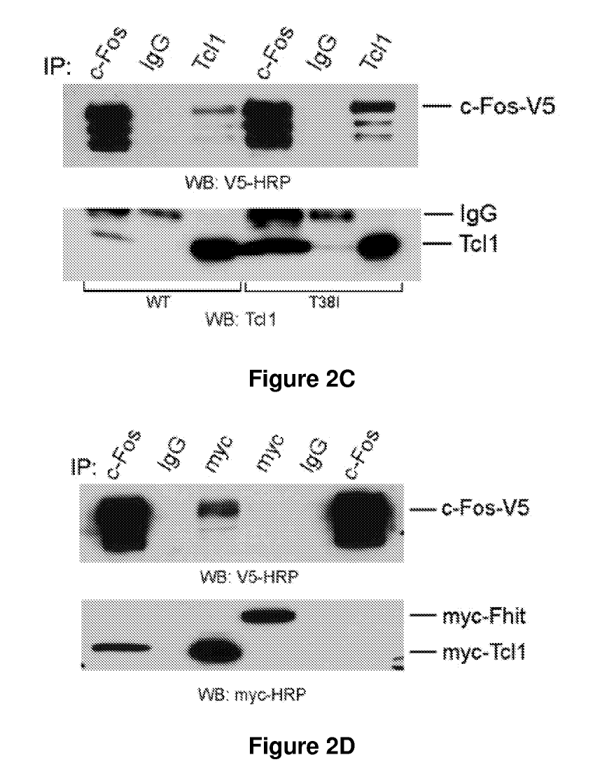

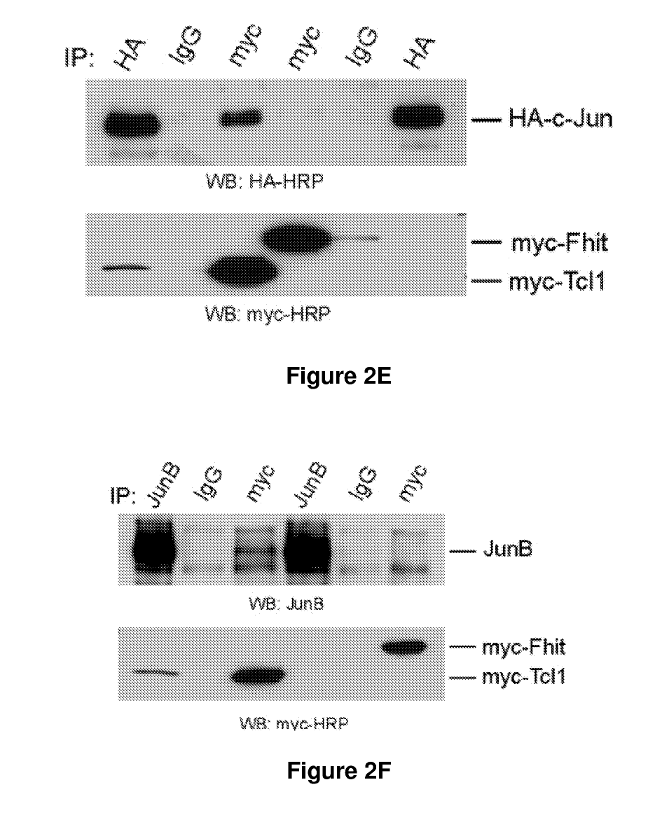

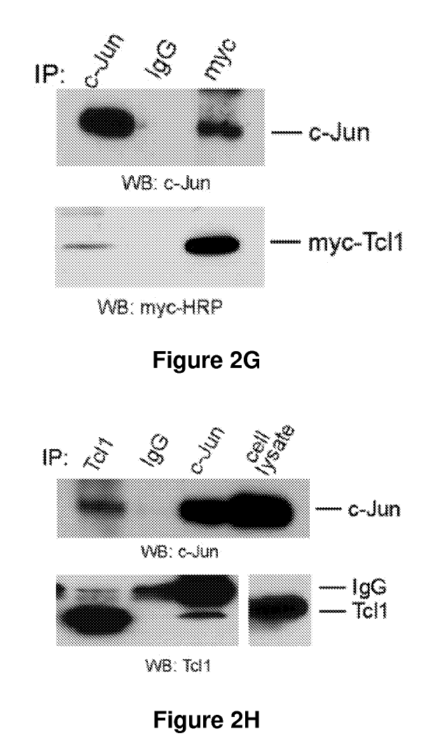

The inventors investigated whether Tcl 1 can inhibit AP1-dependent transcription. To assess the activity of AP-1, a system based on the ability of MEKK1 was used to activate an AP-1 reporter construct, pAP-1-Luc, expressing luciferase under the control of an AP-1-responsive element. Some 293 cells were transfected with the constructs indicated in FIGS. 2A-2H. The inventors herein also investigated whether Tcl1 WT and mutants inhibit the activity of endogenous AP-1 in 293 cells. The 293 cells were transfected with MEKK1 to activate AP-1.

FIG. 2A shows that AP-1 activity was induced 652-fold by MEKK1. Tcl1 expression inhibited AP-1 dependent transactivation ˜2.5-fold, whereas Tcl1 T38I caused a dramatic ˜100-fold inhibition (652 versus 6.3). The R52H mutant also showed a more potent effect compared with WT Tcl 1 (176 versus 287, compared with 652). Similar results were obtained with cells treated with wortmannin (FIG. 2A). Tcl 1 expression inhibited AP-1-dependent transactivation −2.5-fold, whereas the T38I mutant caused 150-fold inhibition (981 versus 6.5). These results indicate that inhibition of AP-1 by Tcl1 is Akt independent. To determine whether Tcl1 inhibits individual components of the AP-1 complex, similar experiments were carried out using WT Tcl1 and the T38I mutant. AP-1 was activated by overexpression of single AP-1 components rather than by using MEKK1.

FIG. 2B-Left shows that Tcl1 inhibits separately c-Fos, c-Jun, and Jun-B, whereas Tcl 1 T38I mutant inhibited c-Fos, c-Jun, and Jun-B −2-fold more effectively. Similar results were obtained with c-Jun/c-Fos and JunB/c-Fos heterodimers (FIG. 2B-Right).

In all of these cases, Tcl1 T38I mutant inhibited more potently than WT Tcl 1. These results (FIG. 2A and FIG. 2B) strongly indicate that Tcl1 mutants show gain-of-function effect in AP-1 inhibition.

To elucidate the mechanism of this inhibition, a series of coimmunoprecipitation experiments were carried out. FIGS. 2C-2F show results of these experiments using transiently expressed proteins. T38I mutant protein showed much robust coimmunoprecipitation with c-Fos than WT Tcl1 (FIG. 2C—Lower vs. FIG. 2C—Upper), suggesting a relation with its more potent inhibition of AP-1 compared with WT Tcl 1. The specificity of this interaction is shown in FIG. 2D; Tcl1 was coimmunoprecipitated with c-Fos in both directions, whereas no positive coimmunoprecipitates were detected between Fhit (used as a negative control) and c-Fos (FIG. 2D-Lower vs. FIG. 2D-Upper).

Similarly, Tcl1 but not Fhit was coimmunoprecipitated with c-Jun (FIG. 2E), and Tcl 1 but not Fhit was coimmunoprecipitated with JunB (FIG. 2F).

FIG. 2G shows that endogenous c-Jun coimmunoprecipitated with transfected Tcl 1 in 293 cells, whereas Tcl 1 was detected in immune complexes of endogenous c-Jun. Physical interaction of endogenous Tcl1 and c-Jun in Daudi cells is shown in FIG. 2H. Tcl 1 was present in immune complexes of endogenous c-Jun, and c-Jun was coimmunoprecipitated with Tcl 1. In these experiments (FIG. 2G and FIG. 2H), because c-Jun is expressed at very low levels, cells were pretreated with phorbol 12-myristate 13-acetate (PMA) and ionomycin. Such treatment significantly induced c-Jun expression in 293 and Daudi cells. Based at least in part on the results described in FIGS. 2A-2H, the inventors now believe that Tcl1 physically interacts with AP-1 components and functions as an AP-1 inhibitor. The fact that both Tcl 1 mutants identified in B-CLL patients show gain-of-function properties in this pathway suggests the ability of Tcl1 to inhibit AP-1-dependent transcription is critical in the pathogenesis of B-CLL.

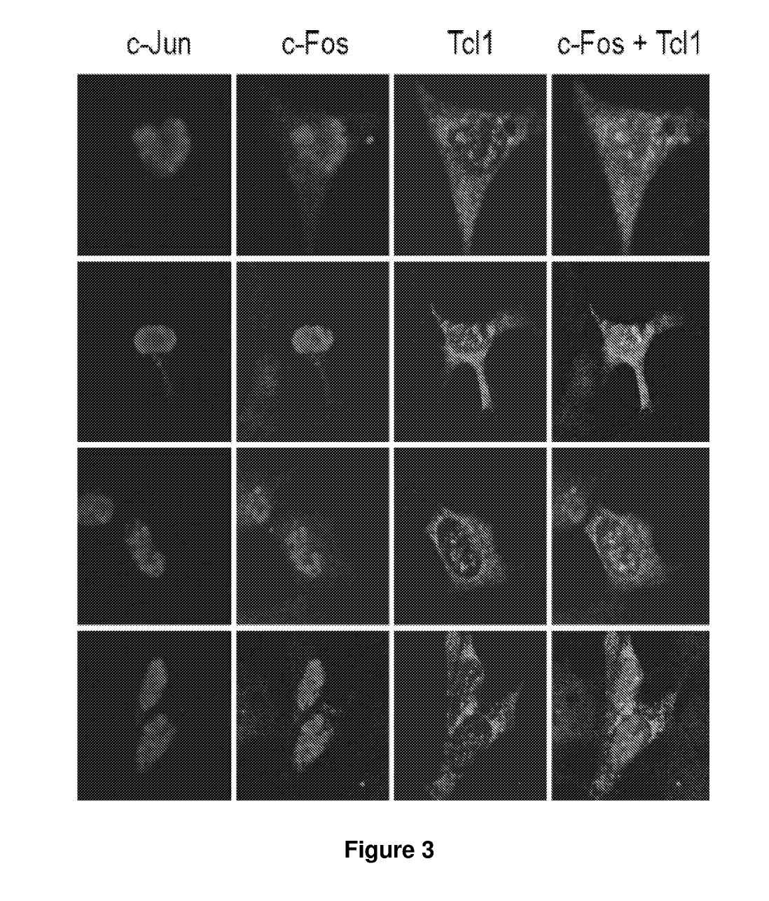

Tcl 1 localizes in both nucleus and cytoplasm. However, c-Jun and c-Fos are mostly nuclear proteins. To determine intracellular localization of Tcl1-AP-1 complexes, immunofluorescence experiments were carried out in 293 cells. FIG. 3 shows intracellular location of Tcl1, c-Jun, and c-Fos in 4 different fields. c-Jun (blue) and c-Fos (red) were colocalized in the nucleus. Tcl1 (green), however, was localized in the nucleus and the cytoplasm. FIG. 3-Right shows that Tcl1-AP-1 complexes (yellow) localized in distinct compartments within the nucleus. These data serve as additional evidence that Tcl 1 inhibits AP-1 function by direct association.

Tcl 1 induces NF-κB-dependent transcription and represses AP-1-dependent transcription by participating directly in transcriptional complexes (FIG. 1 and FIG. 2); as such, the inventors herein now believe that these actions of Tcl1 will result in cell death inhibition. Since MEKK1 induces apoptosis in 293 cells by c-jun N-terminal kinase (JNK) and AP-1 activation, the inventors used the construct expressing the kinase domain of MEKK1 that was used to induce AP-1 in FIG. 2.

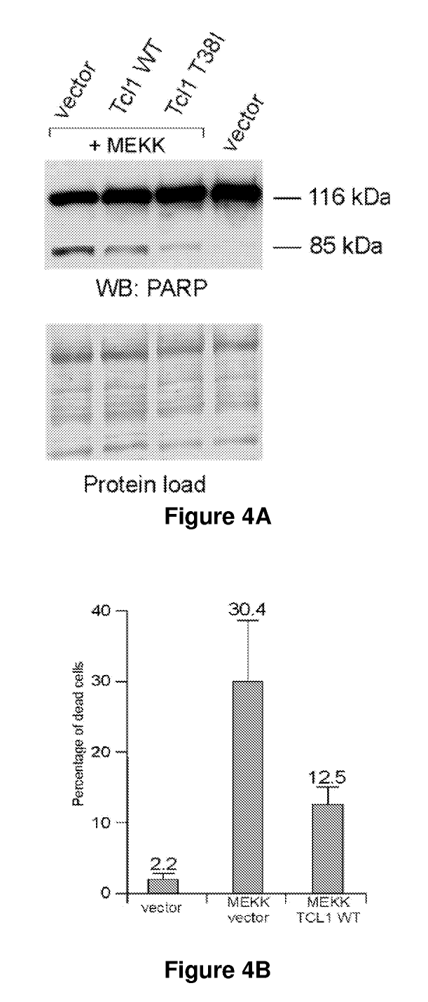

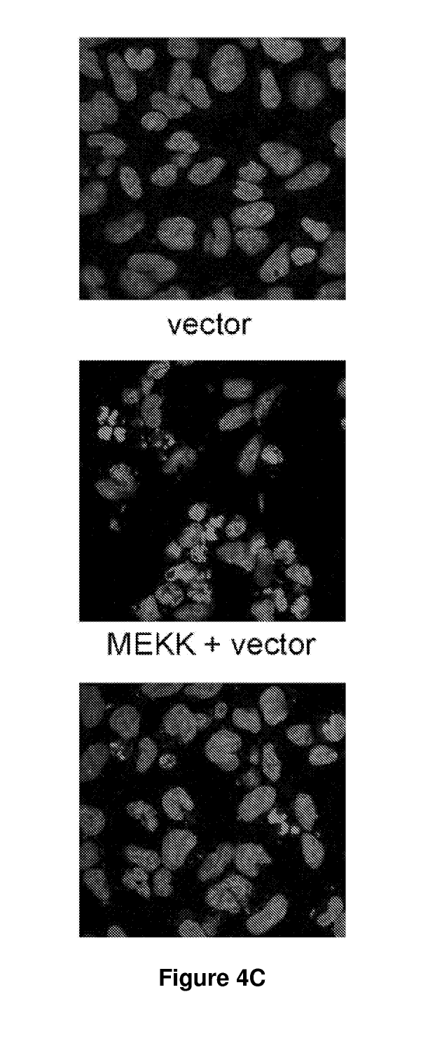

FIGS. 4A-C show that Tcl1 indeed inhibits AP-1-mediated apoptosis in 293 cells. The 116-kDa intact form of poly(ADP-ribose) polymerase 1 (PARP1) is present in both apoptotic and nonapoptotic cells, whereas the 85-kDa cleaved PARP1 isoform is present only in apoptotic cells. Expression of MEKK1 resulted in the appearance of cleaved 85-kDa PARP1 (FIG. 4A).

Tcl 1 expression caused decreased intensity of the 85-kDa band, whereas expression of Tcl1 T38I mutant resulted in a further decrease in expression of 85-kDa PARP1 (FIG. 4A). This finding shows that Tcl 1 inhibits MEKK1-induced apoptosis in 293 cells, whereas expression of the Tcl1 T38I mutant results in even stronger inhibition. To evaluate the number of apoptotic cells, the number of fragmented nuclei was assessed in 293 cells 20 h after transfection. FIG. 4B and FIG. 4C show that MEKK1 transfection resulted in 30% apoptosis in 293 cells, whereas Tcl1 expression resulted in a decrease of apoptosis to 12.5%. These results suggest that Tcl1 inhibits apoptosis caused by AP-1 activation.

Tcl1 functions as an AP-1 inhibitor, thus providing important insights concerning molecular mechanisms involved in B-CLL development. The importance of these results is greatly enhanced by the fact that the somatic T38I mutant showed gain-of-function properties. The R52H mutation was present in constitutional DNA of the same patient and also led to gain of function in AP-1 inhibition. While not wishing to be bound by theory, the inventors herein believe that this change represents a rare polymorphism causing genetic predisposition to B-CLL. The physical interaction between Tcl1 and transcription factors such as p300 and AP-1 components provides a novel molecular mechanism of Tcl1 function and proves that this function of Tcl1 is independent of Akt.

Further, since Tcl1 binds to multiple proteins of different structure and function (such as Akt, p300, c-Jun, and c-Fos), the inventors herein now believe that Tcl1 has other functions (for example, as a transporter).

The inventors' discovery described herein now shows that methods which inhibit NF-κB or activate AP-1 may be useful in treatment of the aggressive form of B-CLL.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

### Examples

### Methods

B-CLL Samples, Genomic Sequencing, and RT-PCR.

A total of 600 B-CLL samples were obtained after informed consent from patients diagnosed with B-CLL from the CLL Research Consortium. Research was performed with the approval of the Institutional Review Board of Ohio State University. Briefly, blood was obtained from CLL patients, and lymphocytes were isolated through Ficoll/Hypaque gradient centrifugation (Amersham) and processed for RNA extraction by using the standard TRlzol method.

Oligonucleotides used in genomic DNA PCR and sequencing were:

| [SEQ ID NO: 1] | |
|---|---|
| TCL1_149F, | 5′-CATGCTGCCCGGATATAAAG-3′; |
| [SEQ ID NO: 2] | |
| TCL1_539R, | 5′-TGCCTGGAGAACTCCTATTCAT-3′; |
| [SEQ ID NO: 3] | |
| TCL13 1 F, | 5′-GAAGTGAGCTTCAGGGAACAGT-3′;; |
| and | |
| [SEQ ID NO: 4] | |
| CL1_880R, | 5′-ACAGCCACTGTGGACTAAGAGG-3′. |

Oligonucleotides used in RT-PCR and sequencing were:

| [SEQ ID NO: 5] | |
|---|---|
| TCL1D5, | 5′-CCTGTGGGCCTGGGAGAAGT-3′ |
| and | |
| [SEQ ID NO: 6] | |
| TCL1R5, | 5′-TCCTCCACGCCGTCAATCTT-3′. |

DNA Constructs.

Full-length human TCL1 and FHIT ORFs were cloned into a pcDNA4-HisMaxC vector (Omni-Tcl 1, Omni-Fhit, respectively) (Invitrogen) using standard protocols. The full-length human TCL1 ORF was also cloned into a pCMV5 vector to obtain pCMV5-TCL1 WT construct. pCMV5-TCL1 T381 and pCMV5-TCL1 R52H constructs were created by using a standard PCR-based mutagenesis kit from Stratagene. WT and mutant TCL1 and FHIT ORFs were cloned into the pCMV-2×Myc vector, modified from the pCMV-Myc vector (BD Biosciences) with an added Myc tag, creating Myc tags at both 5′ and 3′ termini. The resulting constructs were named 2×Myc-Tcl1 WT, 2×Myc-Tcl 1 T381, and 2×Myc-Fhit.

Mammalian expression constructs for c-Jun and JunB (in pCMV-SPORT6 vector) were purchased from ATCC. c-Jun-HA was constructed by inserting the c-Jun ORF into the pCMV-HA vector (BD Biosciences). The c-Fos-V5 construct was purchased from Invitrogen. The p300-HA construct was purchased from Upstate Biotechnology. The Akt-HA construct has been described previously (Pekarsky Y., et al. (2000) Tcl1 enhances Akt kinase activity and mediates its nuclear translocation; Proc Nat/Acad Sci USA 97:3028-3033). The Dual-luciferase Reporter Assay System and Renilla luciferase reporter vector pRL-TK were purchased from Promega. The AP-1 reporter construct, pAP1-Luc, NF-κB reporter construct, pNF-kB-Luc and the construct encoding the kinase domain of MEKK1 under control of the CMV promoter, pFC-MEKK, were purchased from Stratagene.

Cell Culture, Transfection, Western Blot Analysis, and Immunoprecipitation.

NIH 3T3 and 293 cells were grown in RPMI medium 1640 with 10% FBS and 100·sg/L gentamicin at 37° C. FuGene 6 transfection reagent and protease inhibitor mixture tablets were obtained from Roche. Transfections, except luciferase assay experiments, cell lysate preparations, and Western blot analysis were carried out. Immunoblots were developed by using Pierce ECL Western blot analysis substrate or SuperSignal West Femto Maximum Sensitivity Substrate from Thermo Scientific. Antibodies used were: anti-Tcl1 (sc-32331 for Western blot analysis and immunoprecipitation with p300; sc-11156 and sc-11155 for immunoprecipitation with c-Jun), anti-Omni (sc-7270 for immunoprecipitation and Western blot analysis; sc-499 for immunofluorescence), anti-p300 (sc-32244), anti-Myc (9E10), anti-Myc-HRP (9E10), anti-c-Jun (sc-1694 for immunoprecipitation), anti-Jung (sc-8051 for immunoprecipitation; sc-46 for Western blot analysis), anti-c-Fos (sc-447 for immunoprecipitation and immunofluorescence) (Santa Cruz Biotechnology), anti-c-Jun (610326 for Western blot analysis, BD Biosciences), anti-HA (HA.1 1) (Covance), anti-V5-HRP (Invitrogen), rat anti-HA (for immunofluorescence), and anti-HA-HRP (Roche).

Immunofluorescence.

HEK293 cells were grown on human fibronectin Cellware 2-well culture slides (BD Biosciences). Immunofluorescence experiments were carried out with a Zeiss LCM 510 confocal microscope. Secondary antibodies used for immunofluorescence were as follows: goat anti-mouse Alexa Fluor 546 (red), goat anti-rat Alexa Fluor 647 (far-red), and goat anti-rabbit Alexa Fluor 488 (green), all purchased from Invitrogen.

Luciferase Assay.

NIH 3T3 or 293 cells were transfected with the indicated constructs. Firefly and renilla luciferase activities were assayed with the dual luciferase assay system (Promega), and firefly luciferase activity was normalized to renilla luciferase activity, as suggested by the manufacturer. All experiments were carried out in triplicate and repeated 3 times with consistent results.

Cell Death Analysis.

Apoptosis was assessed by scoring the number of cells displaying fragmented nuclei, stained with 10·sg/mL of Hoechst 33342 (Invitrogen). An alternative method of apoptosis detection was also used. HEK 293 cells were transfected with either 1.5 μg of pCMV5-empty vector or 0.5 μg of pFC-MEKK with 1 μg of pCMV5-empty or pCMV5-Tcl1 WT or pCMV5-Tcl 1 T381 constructs. Twenty-four hours later both dead and live cells were collected and lysed. These lysates were probed with anti-PARP1 antibody (556362; BD Biosciences). The 116-kDa intact form of PARP1 was present in both nonapoptotic and apoptotic cells. The 85-kDa PARP1 cleavage fragment was present only in apoptotic cells.

Therapeutic/Prophylactic Methods and Compositions

The invention provides methods of treatment and prophylaxis by administration to a subject an effective amount of a therapeutic, i.e., a monoclonal (or polyclonal) antibody, viral vector, Tcl1 mimic or Tcl1 antagonist of the present invention. In a preferred aspect, the therapeutic is substantially purified. The subject is preferably an animal, including but not limited to, animals such as cows, pigs, chickens, etc., and is preferably a mammal, and most preferably human.

Various delivery systems are known and are used to administer a therapeutic of the invention, e.g., encapsulation in liposomes, microparticles, microcapsules, expression by recombinant cells, receptor-mediated endocytosis, construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the therapeutic is a nucleic acid encoding a protein therapeutic the nucleic acid is administered in vivo to promote expression of its encoded protein, by constructing it as part of an appropriate nucleic acid expression vector and administering it so that it becomes intracellular, or coating with lipids or cell-surface receptors or transfecting agents, or by administering it in linkage to a homeobox-like peptide which is known to enter the nucleus. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutics of the invention are formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

1.-11. (canceled)

12. An antibody which binds to an epitope on Tcl1, wherein the antibody modulates at least one of: an interaction between the epitope and activator protein 1 (AP-1).

13. A pharmaceutical composition comprising an antibody of claim 12.

14. A method of treating a B-CLL disease state in which the activity of activator protein 1 (AP-1) is altered in a mammal, comprising:
administering to the mammal a therapeutically effective amount of an antibody capable of binding to an epitope on a Tcl1 protein, thereby modulating a Tcl1 enhanced activity of the activator protein 1 (AP-1).

15. A method of treating a B-CLL disease state in which the activity of activator protein 1 (AP-1) is altered in a mammal, comprising:
administering to the mammal a therapeutically effective amount of a peptide fragment of activator protein 1 (AP-1),
wherein the peptide fragment binds to the activator protein 1 (AP-1), thereby modulating a Tcl1 enhanced kinase activity of the activator protein 1 (AP-1).

16. A compound comprising a Tcl1 mimic, wherein the Tcl1 mimic binds to an activator protein 1 (AP-1) in any cell and is functionally active in mimicking a Tcl1 enhanced activation of the activator protein 1 (AP-1).

17. A method of treating a disease state in which the activity of activator protein 1 (AP-1) is altered in a mammal, comprising:
administering to the mammal a therapeutically effective amount of a Tcl1 mimic, wherein the Tcl1 mimic binds to the activator protein 1 (AP-1), thereby activating a Tcl1 enhanced kinase activity of the activator protein 1 (AP-1).

18. A compound comprising a Tcl1 antagonist, wherein the Tcl1 antagonist binds to activator protein 1 (AP-1) in any cell and is functionally active in modulating a Tcl1 enhanced activation of the activator protein 1 (AP-1).


SEQUENCE LISTING (10 sequences)

SEQ ID NO: 1 - DNA, 20 bp, Artificial Sequence
  Description of Artificial Sequence Synthetic oligonucleotide
catgctgcccggatataaag

SEQ ID NO: 2 - DNA, 22 bp, Artificial Sequence
  Description of Artificial Sequence Synthetic oligonucleotide
tgcctggagaactcctattcat

SEQ ID NO: 3 - DNA, 22 bp, Artificial Sequence
  Description of Artificial Sequence Synthetic oligonucleotide
gaagtgagcttcagggaacagt

SEQ ID NO: 4 - DNA, 22 bp, Artificial Sequence
  Description of Artificial Sequence Synthetic oligonucleotide
acagccactgtggactaagagg

SEQ ID NO: 5 - DNA, 20 bp, Artificial Sequence
  Description of Artificial Sequence Synthetic oligonucleotide
cctgtgggcctgggagaagt

SEQ ID NO: 6 - DNA, 20 bp, Artificial Sequence
  Description of Artificial Sequence Synthetic oligonucleotide
tcctccacgccgtcaatctt

SEQ ID NO: 7 - DNA, 15 bp, Artificial Sequence
  Description of Artificial Sequence Synthetic oligonucleotide
cccttaaycatcgag

SEQ ID NO: 8 - protein, 5 aa, Artificial Sequence
  Description of Artificial Sequence Synthetic peptide
PLXIE

SEQ ID NO: 9 - DNA, 15 bp, Artificial Sequence
  Description of Artificial Sequence Synthetic oligonucleotide
ctcttgcrtcgggaa

SEQ ID NO: 10 - protein, 5 aa, Artificial Sequence
  Description of Artificial Sequence Synthetic peptide
LLXRE